SEQUENCE LISTING (4 sequences)

SEQ ID NO: 1 - DNA, 31 bp, Artificial Sequence
  A circa 400 bp fragment of Golgi alpha-Mannosidase II, obtained via RT-PCR from leaf RNA of Lycopersicon esculentum, was inserted twice flanked to the first intron of the potato GA20-Oxidase over SalI/BamHI and/or XhoI/Bg1II via compatible overlaps.
caccgtcgacagtccaagcacatcctagata

SEQ ID NO: 2 - DNA, 28 bp, Artificial Sequence
  A circa 400 bp fragment of Golgi alpha-Mannosidase II, obtained via RT-PCR from leaf RNA of Lycopersicon esculentum, was inserted twice flanked to the first intron of the potato GA20-Oxidase over SalI/BamHI and/or XhoI/Bg1II via compatible overlaps.
nnnggatccaaattctggtttaaagcca

SEQ ID NO: 3 - DNA, 31 bp, Artificial Sequence
  A circa 400 bp fragment of Golgi alpha-Mannosidase II, obtained via RT-PCR from leaf RNA of Lycopersicon esculentum, was inserted twice flanked to the first intron of the potato GA20-Oxidase over SalI/BamHI and/or XhoI/Bg1II via compatible overlaps.
caccggatccaagcacatcctagatatgttg

SEQ ID NO: 4 - DNA, 29 bp, Artificial Sequence
  A circa 400 bp fragment of Golgi alpha-Mannosidase II, obtained via RT-PCR from leaf RNA of Lycopersicon esculentum, was inserted twice flanked to the first intron of the potato GA20-Oxidase over SalI/BamHI and/or XhoI/Bg1II via compatible overlaps.
nnngtcgaccaaattctggtttaaagcca

(19) United States
(12) Patent Application Publication
(10) Pub. No.: US 2012/0036600 A9
(43) Pub. Date: Feb. 9, 2012

(54) METHOD FOR GENERATING HYPOALLERGENIC GLYCOPROTEINS IN MUTATED OR TRANSGENIC PLANTS OR PLANT CELLS, AND MUTATED OR TRANSGENIC PLANTS AND PLANT CELLS FOR GENERATING HYPOALLERGENIC GLYCOPROTEINS

(75) Inventors: Antje Von Schaewen, Muenster (DE); Heidi Kaulfuerst-Soboll, Ascheberg (DE)

(73) Assignee: WESTFAELISCHE WILHELMS-UNIVERSITAET MUENSTER, Muenster (DE)

(21) Appl. No.: 12/990,497

(22) PCT Filed: Apr. 25, 2009
(86) PCT No.: PCT/EP2009/003033

§ 371 Date: Oct. 30, 2010

(30) Foreign Application Priority Data
May 3, 2008 (EP) 08008396.7

(51) Int. Cl. A01H 5/00; C07K 14/415; C12N 5/04; C12N 5/10; C12P 21/00; C12N 9/24

(52) U.S. Cl. 800/298; 435/69.1; 435/70.1; 435/200; 435/410; 435/419; 530/395

(57) ABSTRACT

Method for providing a hypoallergenic glycoprotein includes growing at least one of a mutated plant, a part of the mutated plant, plant cells produced from the mutated plant, a transgenic plant, a part of the transgenic plant and plant cells produced from the transgenic plant wherein an activity of an enzyme Golgi α-mannosidase II has been eliminated or decreased so as to obtain a grown material. The hypoallergenic glycoprotein is isolated from the grown material.

### CROSS REFERENCE TO PRIOR APPLICATIONS

This application is a U.S. National Phase application under 35 U.S.C. §371 of International Application No. PCT/EP2009/003033, filed on Apr. 29, 2009 and which claims benefit to European Patent Application No. 08008396.7, filed on May 3, 2008. The International Application was published in German on Nov. 12, 2009 as WO 2009/135603 A1 under PCT Article 21(2).

### FIELD

The present invention provides a method for generating hypoallergenic glycoproteins in mutated or transgenic plants, parts of these plants or plant cells produced therefrom. The present invention also provides the corresponding mutated or transgenic plants, plant parts and plant cells.

### SEQUENCE LISTING

The Sequence Listing associated with this application (SEQ ID NOs: 1, 2, 3 and 4 MANII-dsRNAi constructs) is filed in electronic form via EFS-Web and hereby incorporated by reference into this specification in its entirety. The name of the text file containing the Sequence Listing is Sequence_Listing_Project_ST25. The size of the text file is 2,388 Bytes, and the text file was created on Oct. 18, 2010.

### BACKGROUND

N-glycosylation in the secretory system is an essential process in all eukaryotes. Glycoproteins are first assembled in the endoplasmic reticulum (ER), wherein membrane-bound glycans (dolichol pyrophosphate oligosaccharides) are cotranslationally transferred to specific asparagine residues in the growing polypeptide chain. In higher organisms, sugar units, which are situated on the surface of the folded polypeptide chain, are subject to further trimming and modification reactions in Golgi vesicles. By means of various glycosidases and glycosyltransferases in the ER and Golgi apparatus (the provision can be different in a species-dependent manner), first typical Glc₃Man₉GlcNAc₂-base units (core glycans) of the high mannose type are formed and then, during passage through the various Golgi vesicles, are converted into what are termed “complex” glycans. The latter are distinguished by a lower number of mannose units and the possession of further sugar residues such as fucose and/or xylose and also galactose in plants or sialic acid (N-acetylneuraminic acid, NeuNAc) in mammals.

In higher plants, the formation of complex N-glycans in the secretory system comprises in total eight steps (FIG. 1), wherein the linking to core α1,3-fucose and β1,2-xylose residues is characteristic of plant glycoproteins. Since, although these modifications also occur in some invertebrates, in mammals only α1,6-linked core fucose residues exist, plant glycoproteins act immunogenically on the latter. This shows that not only the sugar residues as such but also the type of their linkage determines the binding or non-binding of antibodies.

Glycoproteins are important for medicine and research. However, isolation of glycoproteins on a large scale is complex and expensive. The direct use of conventionally isolated glycoproteins is frequently problematic, since individual residues of the glycan components can trigger unwanted side effects when administered as a therapeutic agent. In most cases, the glycan component cannot be omitted, since it contributes especially to the physicochemical properties (such as folding, stability and solubility) of the glycoproteins.

Yeasts have mostly proved to be unsuitable for obtaining glycoproteins for medicine and research since they can only carry out glycosylations for what is termed the high mannose type. Insects and higher plants exhibit glycoprotein modifications which, though “complex”, are different from those in animals, and so glycoproteins isolated from these organisms act immunogenically in mammals (as described in Faye L, Chrispeels M J, Common antigenic determinants in the glycoproteins of plants, molluscs, and insects. Glycoconj. J. 5, pages 245-256, (1988); and in Strasser R, Stadlmann J, Schähs M, Stiegler G, Quendler H, Mach L, Glössl J, Weterings K, Pabst M, Steinkellner H, Generation of glyco-engineered Nicotiana benthamiana for the production of monoclonal antibodies with a homogeneous human-like N-glycan structure, Plant Biotechnol. J. 6, pages 392-402 (2008). Animal organisms having glycosylation defects are usually not viable since the terminal glycan residues, for example, membrane glycoproteins, have biologically signal functions and are indispensable especially for cell-cell recognition during embryonal development. Although mammalian cell lines (Chinese Hamster Ovary (CHO) cells) having defined glycosylation defects exist, culturing them is labor intensive and costly.

Different glycosylation mutants have already been described for mammals at a cell culture level. These mutants are either affected in the biosynthesis of mature oligosaccharide chains at the dolichol pyrophosphate or in the glycan processing or exhibit differences in their terminal sugar residues. Some of these cell lines have a conditional-lethal phenotype or exhibit defects in intracellular protein transport.

The consequences of these defects for the intact organism are difficult to estimate. It has been observed that a change in the pattern of complex glycans on cell surfaces of mammals is accompanied with tumor formation and metastasis formation as described in Li D, Li Y, Wu X, Li Q, Yu J, Gen J, Zhang X L, Knockdown of Mgat5 inhibits breast cancer cell growth with activation of CD4+ T cells and macrophages, J. Immunol. 180, pages 3158-3165 (2008) and work cited therein. Glycosylation mutants therefore occur very rarely in mammals. Defects summarized under the abbreviation HEMPAS (Hereditary Erythroblastic Multinuclearity with a Positive Acidified Serum lysis test) are based either on a deficit of α-mannosidase II in the Golgi apparatus or in lysosomes and/or low contents of the enzyme N-acetylglucosaminyltransferase II (GnTII) and greatly limit the viability of the mutated organisms, as described in Fukuda, M N, HEMPAS disease: genetic defect of glycosylation, Glycobiology 1, pages 9-15, Review (1990). In “knock-out” mice, in which Golgi α-mannosidase II (GMII) is destroyed, an alternative synthetic pathway is taken in such a manner that they are viable but anemic (as described in Chui D, Oh-Eda M, Liao Y F, Panneerselvam K, Lal A, Marek K W, Freeze H H, Moremen K W, Fukuda M N, Marth J D, Alpha-mannosidase-II deficiency results in dyserythropoiesis and unveils an alternate pathway in oligosaccharide biosynthesis, Cell 90, pages 157-167 (1997), and in Moremen K W, Golgi alpha-mannosidase II deficiency in vertebrate systems: implications for asparagine-linked oligosaccharide processing in mammals, Biochim. Biophys. Acta 1573, pages 225-235 (2002)). Patients having mutations in GnTII suffer from carbohydrate deficient glycoprotein syndrome type II (CDGSII) with serious multiple development defects as described in Tan J, Dunn J, Jaeken J, Schachter H, Mutations in the MGAT2 gene controlling complex N-glycan synthesis cause carbohydrate-deficient glycoprotein syndrome type II, an autosomal recessive disease with defective brain development, Am. J. Hum. Genet. 59, pages 810-807 (1996). Knock-out mice, in which the gene for N-acetylglucosaminyltransferase I (GnTI) has been destroyed, die as early as the embryonal stage of multiple development defects (as described in loffe E, Stanley P, Mice lacking N-acetylglucosaminyltransferase I activity die at midgestation, revealing an essential role for complex or hybrid N-linked carbohydrates, Proc. Natl. Acad. Sci. USA 91, pages 728-732, (1994) and; Metzler M, Gertz A, Sarkar M, Schachter H, Schrader J W, Marth J D, Complex asparagine-linked oligosaccharides are required for morphogenic events during post-implantation development, EMBO J. 13, pages 2056-2065 (1994)).

On account of the results with animals or animal cells (complex and susceptible to contamination with human pathogens), in recent years experiments have been increasingly undertaken to generate heterologous glycoproteins in plants or plant cells that are modified in such a manner that they synthesize glycoproteins having decreased immunogenic properties.

For instance, von Schaewen A, Sturm A, O'Neill J, Chrispeels M J, Isolation of a mutant Arabidopsis plant that lacks N-acetyl glucosaminyl transferase I and is unable to synthesize Golgi-modified complex N-linked glycans, Plant Physiol. 102, pages 1109-1118 (1993) describes that Arabidopsis mutants were isolated which are defective in the second step of the glycan modification in the Golgi apparatus, in such a manner that glycoproteins having a uniform Man₅GlcNAc₂ modification accumulate. Despite lack of activity of the N-acetylglucosaminyltransferase I (NAG or GnTI) these Arabidopsis cgl1-mutants, under standardized growth conditions (for example, in climatically controlled chambers or in a greenhouse) do not differ markedly from the wild type. GNTI-coding cDNA sequences from Arabidopsis, potato and tobacco were successfully used for antisense throttling of complex glycoprotein glycans in Solanaceae as described in Wenderoth I, von Schaewen A, Isolation and characterization of plant N-acetyl glucosaminyltransferase I (GntI) cDNA sequences, Functional analyses in the Arabidopsis cgl mutant and in antisense plants, Plant Physiol. 123, pages 1097-1108 (2000). This was a first indication that agronomically important cultured plants in principle tolerate lack of GnTI activity and can be used for producing modified glycoproteins.

Attempts were then made on this basis to develop a model system for producing hypoallergenic glycoproteins in plants. Serving for this purpose by way of example to date has been a stable tobacco line as described in U.S. Pat. No. 6,841,659 in which human glucocerebrosidase (hCG; EC 3.2.1.45) accumulates in the apoplast. Additional GNTI-RNAi throttling in this system led, however, to hCG no longer being detected (FIG. 8).

Shaaltiel Y, Bartfeld D, Hashmueli S, Baum G, Brill-Almon E, Galili G, Dym O, Boldin-Adamsky S A, Silman I, Sussman J L, Futerman A H, Aviezer D, Production of glucocerebrosidase with terminal mannose glycans for enzyme replacement therapy of Gaucher's disease using a plant cell system, Plant Biotechnol. J. 5, pages 579-590 (2007) describes the production of hGC in a carrot cell suspension. The recombinant protein was transported to the vacuoles here with the aid of a C-terminal vacuolar targeting signal (CTTP) and therefore carries immunogenic glycans typical of plants. The system used in this approach additionally has the disadvantage that, for targeting vacuoles, extra sequences had to be used that are not permitted for clinical phase III studies.

Targeted over production of hGC in tobacco seeds resulted in a decrease in the vitality of the recombinant seed material. Although hGC preparations obtained from this seed material have reduced contents of immunogenic xylose and fucose residues, galactose and glucose modifications were detected instead. Although this did not impair the functionality of the enzyme preparation, it decreased the uptake into fibroblasts by Gaucher's disease patient (as described in Reggi S, Marchetti S, Patti T, De Amicis F, Cariati R, Bembi B, Fogher C, Recombinant human acid beta-glucosidase stored in tobacco seed is stable, active and taken up by human fibroblasts, Plant Mol. Biol. 57, pages 101-113 (2005)), wherefore mannose-terminated glycans, as in the present invention, seem to be more suitable (compare anti-PHA-L (phytohemagglutinin L) for ConA binding in FIG. 6).

Strasser R, Stadlmann J, Schähs M, Stiegler G, Quendler H, Mach L, Glössl J, Weterings K, Pabst M, Steinkellner H, Generation of glyco-engineered Nicotiana benthamiana for the production of monoclonal antibodies with a homogeneous human-like N-glycan structure, Plant Biotechnol. J. 6, pages 392-402 (2008) describes that, by means of RNAi throttling in Nicotiana benthamiana, both core fucosyltransferases and also at the same time xylosyltransferase were decimated. Jin C, Altmann F, Strasser R, Mach L, Schähs M, Kunert R, Rademacher T, Glössl J, Steinkellner H, A plant-derived human monoclonal antibody induces an anticarbohydrate immune response in rabbits, Glycobiology 18, pages 235-241 (2008) describes, that the monoclonal antibody generated in these plants had hypoallergenic properties. Glycoproteins from these plants should, similarly to the wild type (FIG. 6), likewise scarcely have any mannose-terminated glycoproteins, which appears to decrease the activity (clearance) of humoral-administered hGC in Gaucher's disease patients.

For medicine and research, there is still a requirement for expression systems and methods for being able to produce recombinant, and in particular, hypoallergenic, glycoproteins inexpensively.

### SUMMARY

An aspect of the present invention is to provide a suitable method and suitable plants or plant materials for generating hypoallergenic glycoproteins, for example, for the therapy of lysosomal storage diseases.

In an embodiment, the present invention provides a method for providing a hypoallergenic glycoprotein which includes growing at least one of a mutated plant, a part of the mutated plant, plant cells produced from the mutated plant, a transgenic plant, a part of the transgenic plant and plant cells produced from the transgenic plant wherein an activity of an enzyme Golgi α-mannosidase II has been eliminated or decreased so as to obtain a grown material. The hypoallergenic glycoprotein is isolated from the grown material.

### BRIEF DESCRIPTION OF THE FIGURES

The present invention is described in greater detail below on the basis of embodiments and of the figures in which:

FIG. 1 shows a sequence of plant-typical N-glycan modifications, to which secretory glycoproteins are subject during passage through the ER and the Golgi apparatus;

FIG. 2 shows a diagram of the RNAi constructs used;

FIG. 3 shows immunoblot detection of the CCD pattern (using PHA-L antiserum) in tomato fruit extracts of the MANII-RNAi primary transformants (T0) with wild type (Wt) and GNTI-RNAi lines for comparison (top);

FIG. 4 shows comparison of hypoallergenic tomato fruits;

FIG. 5 shows CCD patterns in leaf extracts of selected Arabidopsis N-glycosylation mutants and tomato RNAi lines with immunodetection using PHA-L antiserum that recognizes predominantly xylose residues (Laurière et al., 1989) and—after stripping the blot—using HRP antiserum (stronger core fucose recognition);

FIG. 6 shows tomato fruit extracts of wild type and RNAi lines without/with PNGase F treatment;

FIG. 7 shows immunoblot analysis of tomato fruit extracts with patient sera; and

FIG. 8 shows detectability of the hGC signal in dependence on the N-glycan decoration in apoplast eluates from leaves of tobacco RNAi lines.

### DETAILED DESCRIPTION

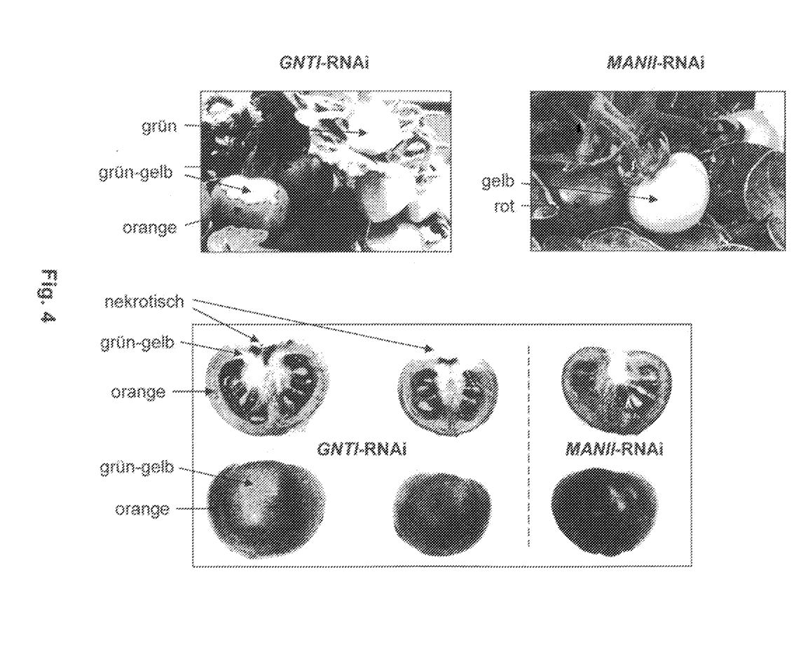

The present inventors have surprisingly found that plants in which the activity of the Golgi α-mannosidase II (GMII, EC 3.2.1.114) is suppressed show a significant reduction of the immunological recognition of proteins having complex glycosylation in transgenic plants and thereby also a general reduction of CCD-allergens (cross-reactive carbohydrate determinants of the structure Man₃XylFucGlcNac₂ or Man₃XylGlcNac₂), for example, generate hypoallergenic glycoproteins in a high extent. Although this was also largely the case in GNTI-RNAi lines studied in parallel, in contrast to the GNTI-RNAi lines, MANII-RNAi lines led to no losses of vitality in the plants, in this case tomatoes (FIG. 4).

This result was completely unexpected since previously described knock out mutants of Golgi α-mannosidase II in Arabidopsis thaliana produced N-glycans which, on the basis of mass spectrometric glycan analyses, were expected to have normal immunogenicity since they carry not only core fucose residues but also xylose residues (as described in Strasser R, Schoberer J, Jin C, Glössl J, Mach L, Steinkellner H, Molecular cloning and characterization of Arabidopsis thaliana Golgi alpha-mannosidase II, a key enzyme in the formation of complex N-glycans in plants, Plant J. 45, pages 789-803 (2006)). The prior art, therefore, made it appear highly unlikely that plants having absent or decreased activity of Golgi α-mannosidase II could be suitable for generating hypoallergenic glycoproteins.

An embodiment of the present invention provides for a method for generating hypoallergenic glycol-proteins comprising growing a mutated or transgenic plant, parts of these plants or plant cells produced therefrom, and isolating a desired hypoallergenic glycoprotein from the material grown, which comprises eliminating or decreasing the activity of the enzyme Golgi α-mannosidase II in the mutated or transgenic plant, parts of these plants or plant cells produced therefrom.

Parts of such plants can also comprise, for example, seeds and propagation material. For simplification, parts of such plants and also plant cells in this application are also termed in summarized form as plant material or plant materials.

Golgi α-mannosidase II (GMII, EC 3.2.1.114) is an enzyme which is localized in the Golgi apparatus of multicelled eukaryotes and can catalyze the hydrolysis of branched mannose residues on the α1,6-arm (for example, of mannoses in both α1,3- and α1,6-linking). When the Golgi α-mannosidase II is inactivated, the mannose-terminated glycans remain on the α1,6-arm and are not eliminated, which is of importance for the present invention.

Methods for eliminating or decreasing the activity of Golgi α-mannosidase II which come into consideration are effective gene silencing approaches, for example cosuppression, antisense or RNAi throttling as described in Waterhouse P M, Smith N A, Wang M B; Virus Resistance and Gene Silencing: Killing the Messenger, Trends Plant Sci. 4, pages 452-457 (1999). However, what are likewise termed knock out mutants, in which the gene(s) which encodes/encode Golgi α-mannosidase II have been, in a targeted manner, eliminated or made non-functional, or plants modified in another way that do not possess intact Golgi α-mannosidase II, can be used.

The plants or plant parts or plant cells used in the method provided in the present invention can be transformed in advance with the gene that encodes the desired glycoprotein. Methods for introducing such genes into plants are known to those skilled in the art and are part of the general prior art; for example, either by T-DNA transfer by means of agrobacteria or by direct gene transfer into protoplasts by means of electroporation or PEG, and also by relatively new biolistic methods after bombarding plant cells in whole tissues with DNA-encased metal spheres such as, for example, a gene gun from BIORAD.

The hypoallergenic glycoprotein generated can, for example, be a therapeutic protein such as glucocerebrosidase (glucosylceramidase; D-glucosyl-N-acylsphingosin-glucohydrolase, EC 3.2.1.45), or human glucocerebrosidase (hCG), a glycoprotein for treating Gaucher's disease, the uptake of which into liver cells of the patients is based on terminal mannose residues as described in Barton N R, Furbish F S, Murray G J, Garfield M, Brady R O, Therapeutic Response to Intravenous Infusions of Glucocerebrosidase in a Patient with Gaucher Disease, Proc. Natl. Acad. Sci. USA. 87, pages 1913-1916 (1990). The hypoallergenic glycoprotein can likewise be another secreted glycoprotein therapeutic agent such as, for example, an antibody, an interleukin, an interferon, a lipase etc., or a therapeutic agent for a lysosomal storage disease. Production of secreted versions of membrane-anchored enzymes, for example α-mannosidases (GMII itself or lysosomal α-mannosidase) would also be conceivable for treating disorders with HEMPAS syndrome as described in Fukuda M N, HEMPAS Disease: Genetic Defect of Glycosylation, Glycobiology 1, pages 9-15, Review (1990) or GnTII for treatment of CDGSII (carbohydrate deficient glycoprotein syndrome type II) as described in Tan J, Dunn J, Jaeken J, Schachter H, Mutations in the MGAT2 gene controlling complex N-glycan synthesis cause carbohydrate-deficient glycoprotein syndrome type II, an autosomal recessive disease with defective brain development, Am. J. Hum. Genet. 59, pages 810-807 (1996).

In an embodiment of the present invention, those plants or plant materials can be used in which the heterologous glycoprotein accumulates in the apoplast/the cell wall or in vacuoles. This simplifies the purification from leaves, since glycoprotein preparations enriched in this manner contain fewer impurities. See, for example, U.S. Pat. No. 6,841,659.

Suitable plants in the method provided in the present invention are, for example, the genetic model plant Arabidopsis thaliana or Solanaceae such as, for example, tomato plants (Lycopersicon spec.), tobacco plants (Nicotiana spec.) or potato plants (Solanum spec.). In addition, other agronomically important plants such as, for example, rice and corn are suitable. In addition, suitable expression systems include duckweed Lemna spec. as described in Cox K M, Sterling J D, Regan J T, Gasdaska J R, Frantz K K, Peele C G, Black A, Passmore D, Moldovan-Loomis C, Srinivasan M, Cuison S, Cardarelli P M, Dickey L F., Glycan Optimization of a Human Monoclonal Antibody in the Aquatic Plant Lemna minor, Nat. Biotechnol. 24, pages 1591-1597 (2006) and also lower plants. For the moss Physcomitrella patens it has been found that targeted knock out strategies are possible owing to homologous recombination as described in Decker E L, Reski R, Moss Bioreactors Producing Improved Biopharmaceuticals, Curr. Opin. Biotechnol. 18, pages 393-398, Review, (2007).

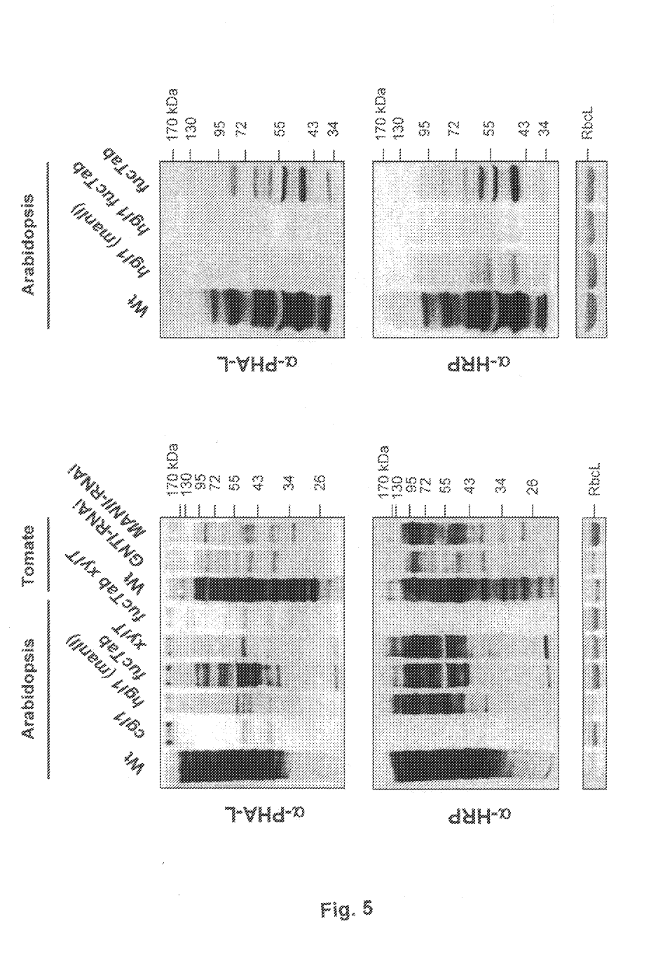

In an embodiment of the present invention, the activity of core fucosyltransferases can, for example, be additionally eliminated or decreased. Enzymes that catalyze the transfer of core α1,3-fucose residues to glycoprotein glycans, such as core α1,3-fucosyl-transferases, are likewise localized in the Golgi apparatus as described in Sturm A, Johnson K D, Szumilo T, Elbein A D, Chrispeels M J, Subcellular localization of glycosidases and glycosyltransferases involved in the processing of N-linked oligosaccharides, Plant Physiol. 85, pages 741-745 (1987), and require at least one terminal GlcNac, i.e., can operate at the earliest subsequently to GnTI as described by Johnson K D, Chrispeels M J, Substrate specificities of N-acetylglucosaminyl-, fucosyl-, and xylosyltransferases that modify glycoproteins in the Golgi apparatus of bean cotyledons, Plant Physiol. 84, pages 1301-1308 (1987). After, by crossing in Arabidopsis, in addition to the activity of GMII (as described by Strasser R, Schoberer J, Jin C, Glössl J, Mach L, Steinkellner H, Molecular cloning and characterization of Arabidopsis thaliana Golgi alpha-mannosidase II, a key enzyme in the formation of complex N-glycans in plants, Plant J. 45, pages 789-803 (2006)) the activity of two core fucosyltransferase isoforms (FucTa and FucTb as described in Strasser R, Altmann F, Mach L, Glössl J, Steinkellner H, Generation of Arabidopsis thaliana plants with complex N-glycans lacking beta1,2-linked xylose and core alphα1,3-linked fucose, FEBS Lett. 561, pages 132-136 (2004)) was also inactivated, in the present invention, hypoallergenic glycoproteins analogous to cgl1 (having a GnTI defect) were achieved (FIG. 5). This should likewise have a favorable effect on the action of antibodies produced recombinantly in plants (ADCC, compare Cox et al.; Decker & Reski; and Strasser R, Stadlmann J, Schähs M, Stiegler G, Quendler H, Mach L, Glössl J, Weterings K, Pabst M, Steinkellner H, Generation of glyco-engineered Nicotiana benthamiana for the production of monoclonal antibodies with a homogeneous human-like N-glycan structure, Plant Biotechnol. J. 6, pages 392-402 (2008)).

The present invention also provides mutated or transgenic plants, parts of these plants or plant cells produced therefrom, wherein the activity of the enzyme Golgi α-mannosidase II is eliminated or decreased in the mutated or transgenic plant, the parts of these plants or plant cells produced therefrom and these produce a hypoallergenic heterologous glycoprotein.

In transgenic plants or plant materials, for example, the activity of the enzyme core fucosyltransferase is likewise eliminated or decreased.

The transgenic plants or plant materials, the glycoproteins produced, the methods for eliminating or reducing the activity of α-mannosidase II or core fucosyltransferase can, for example, be as described for the provided method according to the present invention.

The present inventors have, furthermore, found in experiments with tomatoes that plants having a decreased Golgi α-mannosidase II activity do not exhibit any impairments in appearance and form completely ripe red fruits without any peculiarities. In contrast, in corresponding plants in which, instead of the activity of α-mannosidase II, the activity of GntI was decreased, marked phenotypes were observed during fruit ripening. Particularly marked in this case were ripeness-inhibited spots and necrotic stalk attachments (compare FIG. 4) which initiated premature fruit drop. Together with an increased pathogen susceptibility of the GNTI-RNAi plants, these factors make purification and isolation of heterologous glycoproteins more difficult.

The transgenic plants or plant materials provided according to the present invention are therefore suitable, for example, not only for generating hypoallergenic heterologous glycoproteins, but also hypoallergenic plants which are suitable as foods for allergic persons for whom the CCD (cross-reactive carbohydrate determinants) epitopes are a problem. The present invention therefore also provides hypoallergenic plants and plant materials as such.

The suitability can be increased by, in addition to the activity of Golgi α-mannosidase II, also eliminating, inhibiting or decreasing the activity of core fucosyltransferase.

Finally, the glycoproteins which are obtainable by means of the method provided according to the present invention or from the plants or plant materials provided according to the present invention are also part of the present invention.

The present invention will now be described in more detail on the basis of exemplary embodiments with reference to the accompanying figures. The exemplary embodiments, however, are not intended to restrict the scope of the present invention.

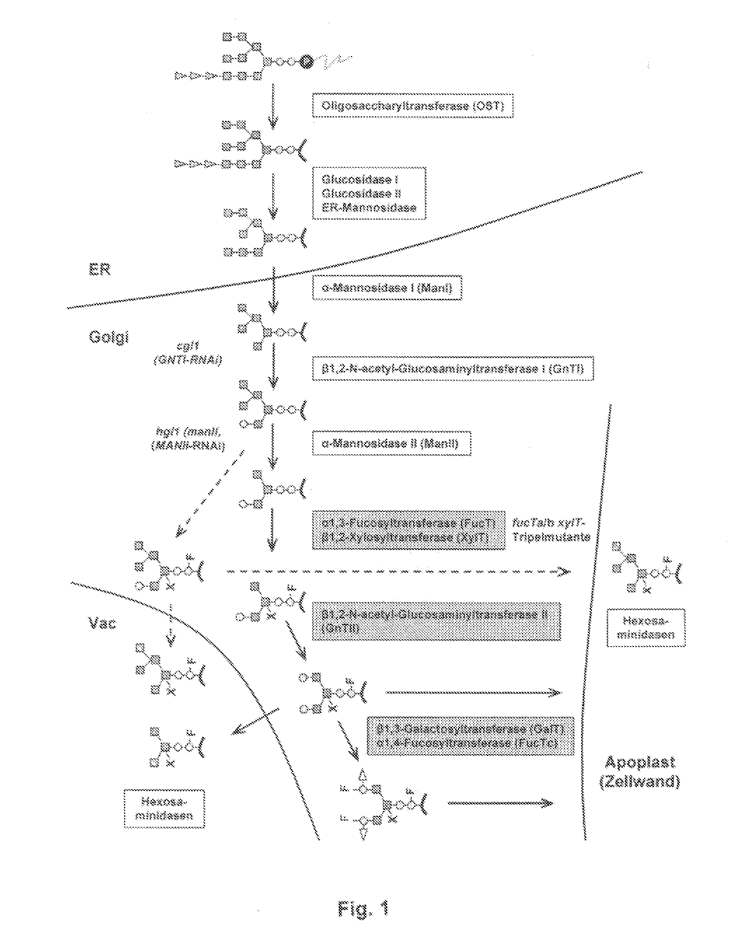

FIG. 1: shows a sequence of plant-typical N-glycan modifications, to which secretory glycoproteins are subject during passage through the ER and the Golgi apparatus. Glycans which accumulate in the event of defects in α-mannosidase II are shown as a pathway with dashed arrows.

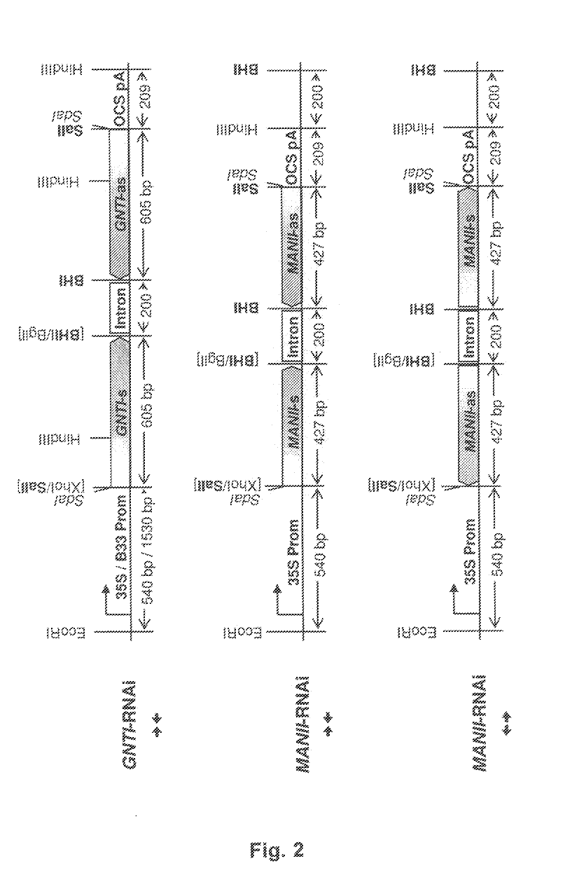

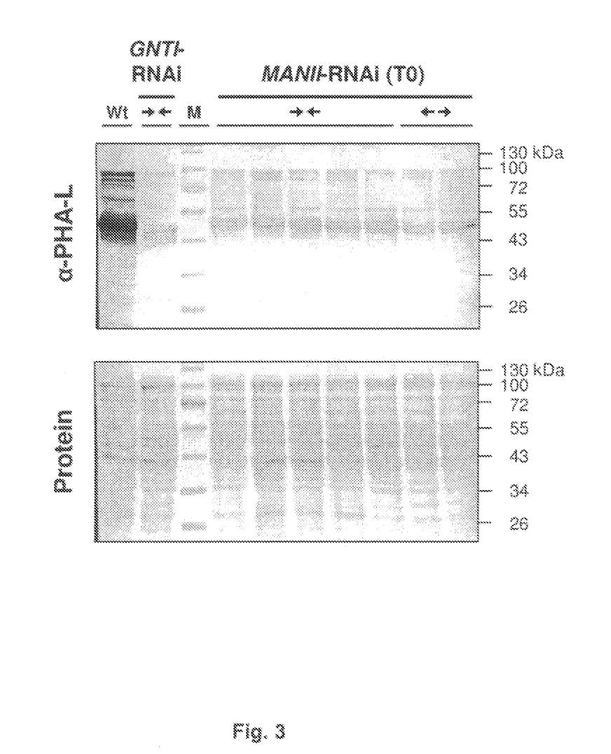

FIG. 2: shows a diagram of the RNAi constructs used. For the MANII-RNAi approach, different orientations of the cDNA fragments were tested in sense (s) and antisense (as) relative to the central intron (result, compare FIG. 3), the dsRNAi construct was cloned in this case in a similar manner described in Chen S, Hofius D, Sonnewald U, Bornke F, Temporal and spatial control of gene silencing in transgenic plants by inducible expression of double-stranded RNA, Plant J. 36, pages 731-740 (2003), with subsequent insertion into the plant expression cassette as described in von Schaewen A, Stitt M, Schmidt R, Sonnewald U, Willmitzer L, Expression of a yeast-derived invertase in the cell wall of tobacco and Arabidopsis plants leads to accumulation of carbohydrate and inhibition of photosynthesis and strongly influences growth and phenotype of transgenic tobacco plants, EMBO J. 9, pages 3033-3044 (1990). Abbreviations: 35S, promoter from cauliflower mosaic virus (constitutive); B33, promoter for Solanaceae (tuber/fruit-specific); OCS pA, polyadenylation signal of octopin synthase (from Agrobacteria T-DNA).

FIG. 3: shows immunoblot detection of the CCD pattern (using PHA-L antiserum) in tomato fruit extracts of the MANII-RNAi primary transformants (T0) with wild type (Wt) and GNTI-RNAi lines for comparison (top). The reversibly Ponceau S-stained membrane (bottom) shows that comparable amounts of protein were transferred. M, marker proteins (pre-stained protein ladder, Fermentas). This shows for the present invention that the CCD throttling in MANII-RNAi lines is comparable with that in GNTI-RNAi lines (with different running behavior of the remaining CCD-reactive glycoproteins).

FIG. 4: shows comparison of hypoallergenic tomato fruits. Whereas GNTI-RNAi lines exhibit a ripeness phenotype (speckled unripe points and necrotic stalk attachments), MANII-RNAi lines form completely ripe fruits. This shows for the present invention that the CCD throttling in MANII-RNAi lines does not impair fruit development phenotypically.

FIG. 5: shows CCD patterns in leaf extracts of selected Arabidopsis N-glycosylation mutants and tomato RNAi lines with immunodetection using PHA-L antiserum that recognizes predominantly xylose residues (as described in Laurière M, Laurière C, Chrispeels M J, Johnson K D, Sturm A, Characterization of a xylose-specific antiserum that reacts with the complex asparagine-linked glycans of extracellular and vacuolar glycoproteins, Plant Physiol. 90, pages 1182-1188 (1989)) and, after stripping the blot, using HRP antiserum (stronger core fucose recognition). Comparison of the two left-hand blots shows for the Arabidopsis mutants that hgl1 (manII) is comparable to xylT and indicates that residual reactivity of the tomato MANII-RNAi extracts must be based on core fucoses. On the right-hand side, after crossing hgl1 (manII) and fucTa fucTb, this gives a further reduction of the CCD recognition in Arabidopsis manII fucTab-triple mutants (without core fucose residues). As the blot development using HRP antiserum (bottom) shows, this implies for the present invention that additional throttling of core fucosyltransferase activity should further reduce the CCD reactivity of the MANII-RNAi lines.

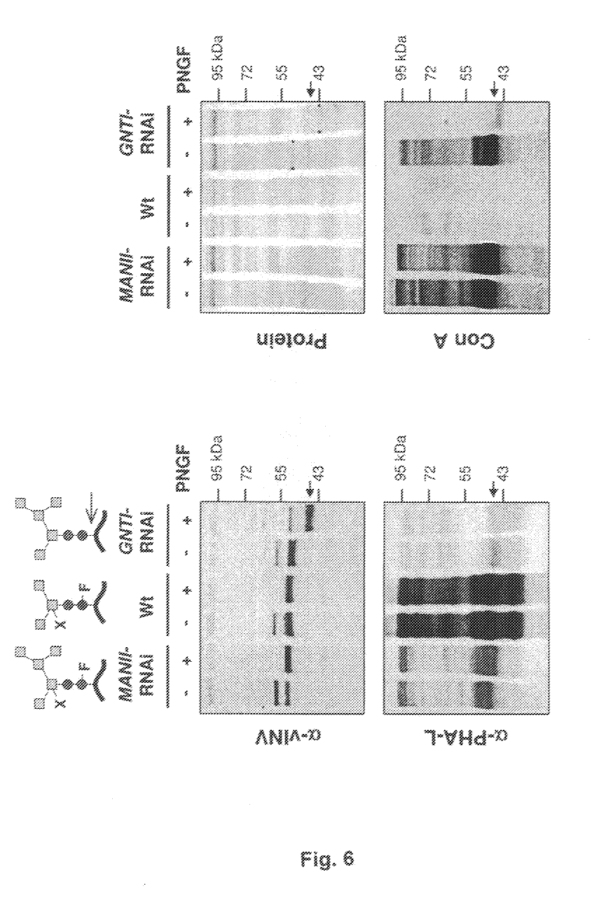

FIG. 6: shows tomato fruit extracts of wild type and RNAi lines without/with PNGase F treatment. The glycoproteins of the MANII-RNAi lines (example: vacuolar invertase, anti-vINV) carry core fucose residues (PNGase F cannot cleave) and untrimmed mannose residues on the α1,6-mannose arm, which results in stronger ConA binding in comparison with the wild type. This shows for the present invention that glycoproteins from hgl1 (manII) mutants or MANII-RNAi lines are suitable, for example, for an uptake into cells of patients having lysosomal storage diseases (such as, for example, Gaucher's disease).

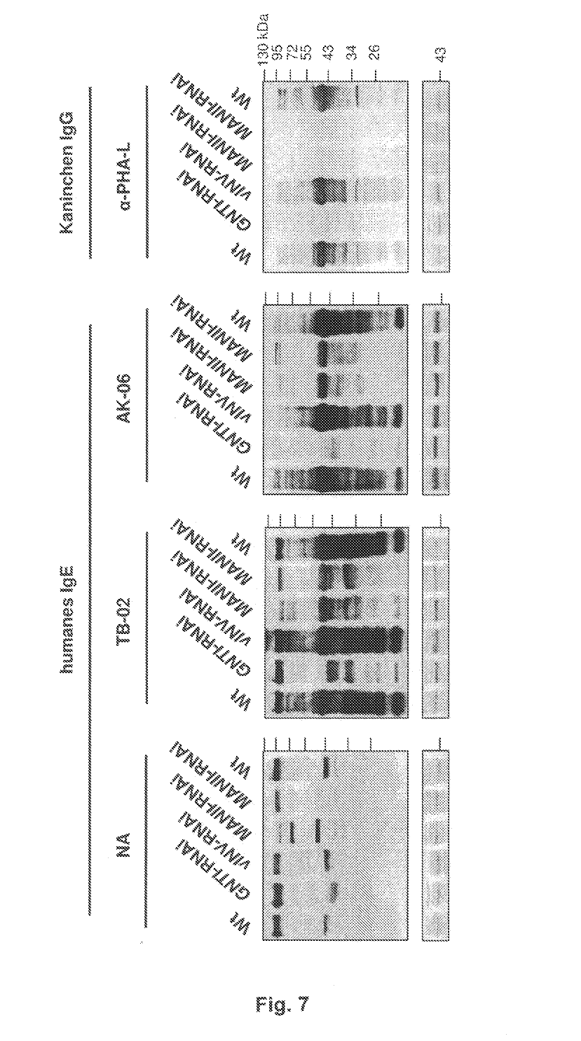

FIG. 7: shows immunoblot analysis of tomato fruit extracts with patient sera. The chemiluminescent detection of allergy-type I-relevant immunoglobulins (IgE) shows reduced reactivity of the two CCD allergy patients with proteins of MANII-RNAi lines which are comparable to GNTI-RNAi lines. Colorimetric over-development of the non-allergic (NA) blot with PHA-L antiserum (rabbit IgG) gives a comparable pattern (control). Below, for comparison of the protein quantities, 43 kDa region of the Ponceau S-stained blot. Abbreviations: NA, non-allergic; TB-02 and AK-06, CCD-allergic patients. This shows for the present invention that CCD-throttling via MANII-RNAi is immunologically relevant for CCD-allergic patients.

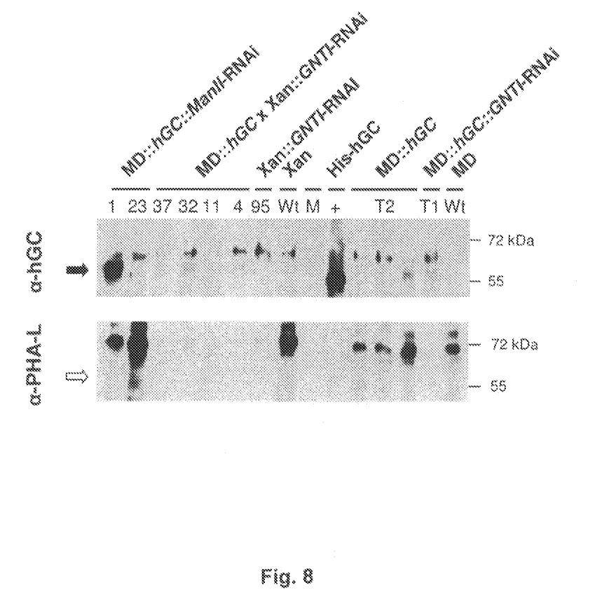

FIG. 8: shows detectability of the hGC signal in dependence on the N-glycan decoration in apoplast eluates from leaves of tobacco RNAi lines. Two double points represent stable integration of the respective constructs into the plant genome. X, progeny which result from crossing the corresponding lines. MD and Xan (Xanthi) designate tobacco cultivars (Nicotiana tabacum). His-hGC, recombinant human glucocerebrosidase with N-terminal His tag without N-glycans (after overexpression in E. coli and subsequent affinity purification). The arrows mark the position of the plant-produced hGC-glycoproteins on the blot which are only significantly detectable in the hypoallergenic MANII-RNAi line no. 1. For the present invention, the lack of binding of PHA-L antiserum shows that hGC carries hypoallergenic N-glycans in MANII-RNAi lines.

### EXAMPLES

### Example 1

### Generation of Tomato Fruits Having Decreased Activity of Golgi α-Mannosidase II (MANII-RNAi Lines)

Production of RNAi lines: the MANII-dsRNAi constructs were designed especially for tomato on the basis of a tomato-EST clone (TA33056_{—}4081 from the TIGR database). An approximately 400 bp fragment of Golgi α-mannosidase II was obtained by means of RT-PCR from leaf RNA of Lycopersicon esculentum of the cultivar Moneymaker “Microtom”. This fragment was inserted into the vector pUC-RNAi (as described in Chen et al., 2003) twice flanking first the intron of potato GA20 oxidase via SalI/BamHI, or XhoI/BglII via compatible overlaps. In this manner two different MANII-dsRNAi constructs were produced. A sense intron-antisense construct having the primers:

(SEQ ID NO: 1)
sense
5′-CACCGTCGACAGTCCAAGCACATCCTAGATA-3′ (SalI
underlined);
and
(SEQ ID NO: 2)
antisense
5′-NNNGGATCCAAATTCTGGTTTAAAGCCA-3′ (BamHI
underlined);

and also an antisense intron-sense construct having the primers:

(SEQ ID NO: 3)
sense
5′-CACCGGATCCAAGCACATCCTAGATATGTTG-3′ (BamHI
underlined);
and
(SEQ ID NO: 4)
antisense
5′-NNNGTCGACCAAATTCTGGTTTAAAGCCA-3′ (SalI
underlined).

Subsequently, the dsRNAi regions are excised using PstI and inserted into an expression cassette (between the constitutive CaMV 35S promoter and the OCS polyadenylation signal) of the SdaI-opened binary vector pBinAR (HygR; as described in Becker, D, Binary vectors, which allow the exchange of plant selectable markers and reporter genes, Nucl. Acids Res. 18, page 203 (1990)).

Using the binary plasmid constructs, competent GV2260 agrobacterial cells (strain C58C1 with virulent plasmid pGV2260, as described in Deblaere R, Bytebier B, De Greve H, Debroeck F, Schell J, van Montagu M, Leemans J, Efficient octopine Ti plasmid-derived vectors of Agrobacterium-mediated gene transfer to plants, Nucl. Acids Res. 13, pages 4777-4788 (1985)) were directly transformed (as described in Höfgen R, and Willmitzer L, Storage of competent cells for Agrobacterium transformation, Nucl. Acids Res. 16, page 9877 (1988)) and used for cocultivation of tomato cotyledons on a feeder layer of tobacco BY2 cells, as described in Ling H-Q, Kriseleit D, Ganal M W, Effect of ticarcillin/potassium clavulanate on callus growth and shoot regeneration in Agrobacterium-mediated transformation of tomato (Lycopersicon esculentum Mill.), Plant Cell Rep. 17, pages 843-847 (1998). Regeneration of MANII-RNAi transformants proceeded under selection pressure (10 mg/l of hygromycin B).

The resultant tomato fruits showed no spots or losses of vitality and, in in vitro analyses, proved to be substantially hypoallergenic for some allergic patients (CCD-allergy patients, see FIG. 7).

### Example 2

### Isolation of (Glyco)Proteins from Tomato Fruits

Protein extraction: First the seeds were removed from fresh tomato fruits and the remaining fruit was either ground in liquid nitrogen to a fine powder either completely or separately in fruit flesh and peel and stored at −80° C. in portions for further use. For the extraction, the ground material was extracted with ice cold buffer (either 100 mM HEPES pH 7.5 or 50 mM HEPES pH 7.5 containing 250 mM NaCl) and further additions (2 mM of Na₂S₂O₅, 1 mM Pefabloc SC, SERVA, proteinase inhibitor cocktail, SIGMA, 1:5000) and then centrifuged at 4° C. for 10 min. The supernatants were used for immunoblots after determining the protein content (as described in Bradford M M, A rapid and sensitive method for the quantitation of microgram quantities of protein utilizing the principle of protein-dye binding, Anal. Biochem. 72, pages 248-254 (1976)).

### Example 3

### Study of the Isolated Glycoprotein Using Various Antisera-Immunoblot Analyses

Protein extracts were separated under reducing conditions in 11-15% strength polyacrylamide gels using SDS-PAGE (as described in Laemmli U K, Cleavage of structural proteins during the assembly of the head of bacteriophage T4, Nature 227, pages 680-685 (1970)) and transferred onto nitrocellulose membranes (0.45 μm PROTRAN, Schleicher & Schüll) in a wet cell (Mini-Protean 3 system, Bio-Rad) for 2 hours at 350 mA. On the membrane the proteins were stained and fixed with Ponceau S (0.3% in 3% TCA, SERVA), and after documentation (flatbed scanner) destained again with TBST (20 mM Tris, 150 mM NaCl, 0.1% (v/v) Tween 20, pH 7.4). The blot membranes were subsequently blocked with 2% milk powder in TBST either overnight at 4° C. or at least for 1 hour at RT.

For chemiluminescent developments, the blots were incubated in dilute antisera (1:5000 in TBST, 2% milk powder) with shaking for 2 hours at RT. They were then washed 3 times with TBST and one further incubation was carried out using HRP-conjugated goat-anti-rabbit IgG (Bio-Rad, 1:10 000 in TBST, 2% milk powder) for 1 hour and subsequent washing 3 times. Development and subsequent stripping of the blot membranes proceeded according to the manufacturer's instructions for the ECL Advance Western Blotting Detection Kit (GE Healthcare).

For marking plant protein glycans (CCD), a rabbit antiserum was routinely used which was produced against PHA-L (as described in Laurière M, Laurière C, Chrispeels M J, Johnson K D, Sturm A, Characterization of a xylose-specific antiserum that reacts with the complex asparagine-linked glycans of extracellular and vacuolar glycoproteins, Plant Physiol. 90, pages 1182-1188 (1989)) and recognizes, in addition to core fucose residues, predominantly xylose residues (1:10 000 in TBST, 2% milk powder for 2 hours). Alternatively, a commercial rabbit antiserum against HRP (Sigma) was used (1:20 000 in 40 mM Tris pH 7.4, 300 mM NaCl, 0.1% (v/v) Tween 20, 2% milk powder for 2 hours), in which the core fucose recognition is elevated owing to peculiarities of the HRP glycoprotein in plant extracts (as described in Wuhrer M, Hokke C H, Deelder A M, Glycopeptide analysis by matrix-assisted laser desorption/ionization tandem time-of-flight mass spectrometry reveals novel features of horseradish peroxidase glycosylation, Rapid Commun. Mass Spectrom. 18, pages 1741-1748 (2004) and in Wuhrer M, Balog C I, Koeleman C A, Deelder A M, Hokke C H, New features of site-specific horseradish peroxidase (HRP) glycosylation uncovered by nano-LC-MS with repeated ion-isolation/fragmentation cycles, Biochim. Biophys. Acta 1723, pages 229-239 (2005)).

For labeling vacuolar invertase (vINV) and human glucocerebrosidase (hGC), antisera were used which were obtained after immunizing rabbits with N-terminal shortened versions and His-tag (first cloning of corresponding cDNA fragments in pET16b, followed by IPTG-induced overexpression in E. coli BL21 cells (Novagen) and subsequent affinity purification on Ni-NTA (Qiagen)). All further steps were carried out as described above.

For colorimetric developments, the antisera were used concentrated 10 fold, incubated with HRP-conjugated goat-anti-rabbit IgG (Bio-Rad, 1:3000 in TBST, 2% milk powder) for 1 hour at RT and detected as described by von Schaewen A, Sturm A, O'Neill J, Chrispeels M J, Isolation of a mutant Arabidopsis plant that lacks N-acetyl glucosaminyl transferase I and is unable to synthesize Golgi-modified complex N-linked glycans, Plant Physiol. 102, pages 1109-1118 (1993).

IgE immunoblots: for immunoblot detection of IgE antibodies from human sera, the blocked blot membranes were incubated with dilute patient sera (1:10 in TBST, 2% milk powder) with shaking for 3 hours at RT, washed 3 times with TBST and then incubated for 1 hour with affinity-purified antibody peroxidase-labeled goat-anti-human IgE(ε) (Kirkegaard & Perry Laboratories, MD, USA) (1:10 000 in TBST) and washed as above. The subsequent chemiluminescent development likewise proceeded with the ECL Advance Western Blotting Detection Kit (GE Healthcare) in accordance with the manufacturer's instructions.

The PNGase F treatment of the tomato fruit extracts followed the manufacturer's instructions (Roche). The ConA affinoblot development was carried out in a similar manner as described by Faye L, Chrispeels M J, Characterization of N-linked oligosaccharides by affinobloting with concanavalin A-peroxidase and treatment of the blots with glycosidases, Anal. Biochem. 149, pages 218-224 (1985) with 10 fold lower concentrations of concanavalin A (ConA, Sigma) and HRP (Fluka) for the ECL development.

The present invention is not limited to embodiments described herein; reference should be had to the appended claims.

1-12. (canceled)

13. Method for providing a hypoallergenic glycoprotein, the method comprising:
growing at least one of a mutated plant, a part of the mutated plant, plant cells produced from the mutated plant, a transgenic plant, a part of the transgenic plant and plant cells produced from the transgenic plant wherein an activity of an enzyme Golgi α-mannosidase II has been eliminated or decreased so as to obtain a grown material; and
isolating the hypoallergenic glycoprotein from the grown material.

14. The method as recited in claim 13, wherein the activity of the enzyme Golgi α-mannosidase II was eliminated or decreased by a mutation or a gene silencing.

15. The method as recited in claim 14, wherein the mutation or the gene silencing is at least one of a cosuppression, an antisense or an RNAi throttling.

16. The method as recited in claim 13, further comprising transforming the at least one a mutated plant, a part of the mutated plant, plant cells produced from the mutated plant, a transgenic plant, a part of the transgenic plant and plant cells produced from the transgenic plant with a gene which encodes the hypoallergenic glycoprotein.

17. The method as recited in claim 13 wherein, the hypoallergenic glycoprotein is glucocerebrosidase or another secreted glycoprotein therapeutic agent.

18. The method as recited in claim 13, wherein the hypoallergenic glycoprotein accumulates in at least one of an apoplast, a cell wall and vacuoles of the at least one of a mutated plant, a part of the mutated plant, plant cells produced from the mutated plant, a transgenic plant, a part of the transgenic plant and plant cells produced from the transgenic plant.

19. The method as recited in claim 18, wherein the isolating of the hypoallergenic glycoprotein is from the at least one of an apoplast, a cell wall and vacuoles.

20. The method as recited in claim 13, wherein the at least one of a mutated plant, a part of the mutated plant, plant cells produced from the mutated plant, a transgenic plant, a part of the transgenic plant and plant cells produced from the transgenic plant are derived from at least one of a Solanaceae, rice, corn and Arabidopsis.

21. The method as recited in claim 20, wherein the Solanaceae is at least one of a tomato plant, a potato plant and a tobacco plant.

22. The method as recited in claim 13, further comprising eliminating or decreasing an activity of an enzyme core-fucosyltransferase in the least one of a mutated plant, a part of the mutated plant, plant cells produced from the mutated plant, a transgenic plant, a part of the transgenic plant and plant cells produced from the transgenic plant.

23. A mutated plant, a part of the mutated plant, plant cells produced from the mutated plant, a transgenic plant, a part of the transgenic plant and plant cells produced from the transgenic plant wherein an activity of an enzyme Golgi α-mannosidase II has been eliminated or decreased and which produce a hypoallergenic heterologous glycoprotein.

24. The mutated plant, the part of the mutated plant, the plant cells produced from the mutated plant, the transgenic plant, the part of the transgenic plant and the plant cells produced from the transgenic plant as recited in claim 23, wherein an activity of an enzyme core-fucosyltransferase has been eliminated or decreased.

25. The mutated plant, the part of the mutated plant, the plant cells produced from the mutated plant, the transgenic plant, the part of the transgenic plant and the plant cells produced from the transgenic plant as recited in claim 23, wherein at least one of the mutated plant, the part of the mutated plant, the plant cells produced from the mutated plant, the transgenic plant, the part of the transgenic plant and the plant cells produced from the transgenic plant is hypoallergenic.

26. Method of producing hypoallergenic plants, the method comprising:
growing at least one of a mutated plant, a part of the mutated plant, plant cells produced from the mutated plant, a transgenic plant, a part of the transgenic plant and plant cells produced from the transgenic plant wherein an activity of an enzyme Golgi α-mannosidase II has been eliminated or decreased so as to obtain a grown material; and
using the grown material to produce hypoallergenic plants.

27. The method as recited in claim 26, further comprising eliminating or decreasing an activity of an enzyme core-fucosyltransferase.

28. A hypoallergenic glycoprotein obtained from at least one of:
a) a method comprising:
growing at least one of a mutated plant, a part of the mutated plant, plant cells produced from the mutated plant, a transgenic plant, a part of the transgenic plant and plant cells produced from the transgenic plant wherein an activity of an enzyme Golgi α-mannosidase II has been eliminated or decreased so as to obtain a grown material, and
isolating the hypoallergenic glycoprotein from the grown material;
b) providing at least one of a mutated plant, a part of the mutated plant, plant cells produced from the mutated plant, a transgenic plant, a part of the transgenic plant and plant cells produced from the transgenic plant wherein an activity of an enzyme Golgi α-mannosidase II has been eliminated or decreased and which produce a hypoallergenic heterologous glycoprotein, and
isolating the hypoallergenic glycoprotein from the at least one of a mutated plant, a part of the mutated plant, plant cells produced from the mutated plant, a transgenic plant, a part of the transgenic plant and plant cells produced from the transgenic plant; and
c) growing at least one of a mutated plant, a part of the mutated plant, plant cells produced from the mutated plant, a transgenic plant, a part of the transgenic plant and plant cells produced from the transgenic plant wherein an activity of an enzyme Golgi α-mannosidase II has been eliminated or decreased so as to obtain a grown material,
using the grown material to produce hypoallergenic plants, and
isolating the hypoallergenic glycoprotein from the hypoallergenic plants;
wherein a mannose-terminated glycans is not eliminated on an α1,6-arm.

29. A hypoallergenic glycoprotein obtained as recited in claim 28, wherein an activity of an enzyme core-fucosyltransferase has been eliminated or decreased.
